(19) United States
(12) Patent Application Publication
(10) Pub. No.: US 2003/0165399 A1
(43) Pub. Date: Sep. 4, 2003

(54) Method for eliminating protozoa, in particular free living amoebas in a colonised aqueous flow, a method for treating an aqueous flow by electropulsing, and its application to eliminating protozoa

(76) Inventors: Marie-Christine Vernhes, Lavaur (FR); Pierre-Andre Cabanes, Paris (FR); Justin Teissie, Ramonville St-Ange (FR)

Correspondence Address: YOUNG & THOMPSON, 745 SOUTH 23RD STREET 2ND FLOOR, ARLINGTON, VA, 22202

(21) Appl. No.: 09/975,953

(22) Filed: Oct. 15, 2001

Related U.S. Application Data
(63) Continuation of application No. PCT/FR00/00981, filed on Apr. 14, 2000.

(30) Foreign Application Priority Data
Apr. 15, 1999 (FR) 99/04749

(51) Int. Cl. A61L 2/03

(52) U.S. Cl. 422/23

(57) ABSTRACT

The invention relates to a method for treating an aqueous flow colonised by cells by applying a pulsed electric field, in which a colonised aqueous flow is subjected to an electric field of an intensity of 1 to 30 kV/cm, and to its application to destroying protozoa, in particular free living amoebas. It also concerns a method for destroying protozoa.

The present invention relates to a method for eliminating protozoa colonising an aqueous medium by applying a pulsed electric field to the medium, to a method for treating a colonised aqueous medium by electropulsing, and to its application to eliminating protozoa.

Thermophilic amoebas are termed “free living” when they can reproduce without passing through an intermediate host. Certain free-living species of amoebas can cause diseases in man: they are amoebas from the genus Naegleria (species fowleri), Acantharoeba (several species) and Balainuthia (species mandrillaris). Free living amoebas exist in several forms:

the vegetative or trophozoite form the infectious form, during which they can multiply;

the encysted form, the resistant form, when the external conditions are unfavourable (for example, a temperature of less than 20° C., the presence of oxidising agents, . . . );

and the flagellate form, for certain amoebas only (in particular Naegleria fowleri).

The natural medium for the development of free living amoebas is fresh water. Acanthamoeba appears to be able to survive in seawater, but Naegleria cannot tolerate a salinity of more than 5 g/l. N. fowleri grows and multiplies above a temperature of 20° C. with an optimum temperature of 25° C. to 45° C. Other factors such as the presence of organic particles in suspension, bacteria or nutrients, also appear to be necessary for their multiplication. Finally, the presence of a warm solid interface, particularly if the current is weak, appears to be particularly propitious to their development.

In natural media, in temperate regions, the abundance of amoebas is essentially observed during hot summers. They can also be abundant in artificially heated water (swimming pools, discharges from industrial sites). As an example, natural warm water in Australia has been shown to contain 900 to 1000 N. fowleri per litre. In artificially heated water, such as in a discharge pond from some power stations, the concentrations do not exceed 1.6 per litre. In contrast, power stations with a closed tertiary cooling circuit where the water circulates in a loop (with some makeup, accompanied by a small amount of discharge), concentrations of N. fowleri are higher. Acanthamoeba is routinely found in domestic water and in natural fresh water. Further, human carriers have been directly observed in many countries, in the nasal cavities, the throat or the digestive tract in a small proportion of the populations studied. In contrast, in hot countries, a majority of subjects may present blood antibodies directed against N. fowleri and Acanthamoeba, and it can thus be concluded that exposure to free living amoebas in such populations is wide.

N. fowleri causes meningoencephalitis. The only route to contamination is the nasal mucous, the amoebas then traversing the mucous and osseous barriers to reach the brain. If all risk of contamination via the digestive tract, in particular drinking water, is excluded from all of the reported cases, a large number of cases appear to be associated with exposure in relatively warm water, by bathing or water sports, which may explain why the disease mainly affects children and young people, as they indulge in more water sports. This fact may also correspond to a less well developed immunity towards such protozoa than in the adult. The disease is very rare, as only 180 cases were reported on Jan. 1, 1998 for the whole world. The difference between the ubiquity of the protozoan and the rarity of pathological manifestations it causes is thus striking, despite the fact that it is highly probable that certain cases have escaped diagnosis, simply because of the rarity of the disease and the absence of clinical specificity. The delay between exposure to a contaminated aquatic medium and the appearance of clinical signs appears to be three to five days. Many antibiotic treatments have been tried. The results are disappointing; only six cases of survival have been reported in the literature. Further, various species of the genus Acanthamoeba can cause keratitis, in particular in contact lens wearers (problem with rinsing with non sterile water). Finally, various species of the genus Acanthamoeba and the genus Balamuthia can cause granulomatous encephalitis in persons with deficient immune defences. In that case, the point of entry is usually cutaneous.

Regarding prevention, it should be emphasised that the risk increases exponentially with the degree of colonisation, which explains the dearth of observed cases in the world, but also the possibility of the emergence of grouped cases if one source is highly contaminated (16 deaths were linked to bathing in a swimming pool in the Czech Republic). When metrological surveillance of a medium reveals too high a risk of meningoencaphalitis, treatment is necessary.

Until now, that treatment has essentially been based on chlorination (American lakes) or chloramination, as in Australia in the drinking water system. In France, a demonstration of the multiplication of such amoebas in certain power stations may result in the firm EDF carrying out chlorination during some summers. This method is effective if the levels of residual free chlorine are sufficiently high and if the treatment is continuous. In the case of high flows to be treated, especially untreated water, such treatment produces by-products that are then released into the environment. Since such products may have long term toxicity effects in man, the search for another treatment method would appear to be apposite.

There is a need for a method and apparatus that can efficiently destroy protozoa, in particular free living amoebas, without causing a secondary effect and which permanently functions economically.

Certain effects connected to the application of an electric field to a cellular suspension have already been described: when a cell is placed in an electric field, it distorts the field lies, causing an accumulation of charge on the cell surface. This results in an induced transmembrane potential difference ΔV which is superimposed on the native difference ΔΨ₀ [Bemlhardt J. and Pauly H. (1973): (1)].

The most complete formula used in the case of a field with square wave kinetics and a spherical cell in suspension is as follows [Kinosita and Tsong (1979) (2)]:

ΔV(t)=fg(λ)rE(t) cos θ(1−e^{-t/τp}) eq 1

The expression for this potential difference induced at a point M at time t is a function of:

E: the intensity of the applied electric field;

f: the form factor for the cell (1.5 in the case of a sphere);

g(λ): factor inked to the conductivities of the external and internal media and to that of the mnembrane with membrane permeability λ;

r: the cell radius;

θ: the angle between the macroscopic electric field vector and the normal to the plane of the membrane at the point considered, M;

τₚ: the charge time for the membrane capacity (of the order of one microsecond);

t: time of application of field.

When the pulse duration is much longer than the time to charge the membrane (t>>τₚ), the term (1−e^{-t/τp}) tends towards 1 to give the stationary state of the conventional formula:

ΔV=fg(λ)rE cos θ eq 2

The term in cos θ indicates that for a given field, the amplitude of this potential difference is not identical at every point of the cell. It is a maximum at points facing the electrodes (poles) and reduces along the cell surface to become zero at the equator.

This potential difference generated by the field is added to the native potential difference αΨ₀. This produces a resultant potential difference ΔVᵣ.

ΔVr=ΔΨ₀+ΔV eq 3

For the cellular hemisphere facing the anode, the numerical values of ΔΨ₀ and ΔV add to take into account the vector of the field effect, causing membrane hyperpolarisation. In contrast, for the hemisphere facing the cathode, the numerical values of ΔΨ₀ and ΔV subtract and the membrane undergoes depolarisation.

When this resulting membrane potential difference exceeds a threshold value estimated to be 200-250 mV [Teissié and Tsong (1981): (3)], a pemeabilisation phenomenon is induced [Ho and Mittal (1996): (4)].

The membrane structure responsible for His membrane permeability is unknown at the present time, and the term “transient permeabilisation structure” (TSP) is preferentially used, which is usually expressed by the term “pores”.

If the electropermeablisation conditions are controlled, this pelmeabilisation phenomenon is transient and reversible, and has little or no effect on cellular viability. This property induced by the field can provide direct access to the cytoplasic contents [Mir et al., (1988): (5); Tsong (1991): (6); Hapala, (1997): (7)]. This allows foreign molecules that are naturally non permeating to penetrate and thus modifies the contents either transiently (electrocharging) or permanently, for example in electrotransformation, electroinsertion techniques, etc.

In contrast, under particular drastic electropulsing conditions, electropermneabilisation is an irreversible phenomenon that leads to cell death, or electromortality [Sale and Hamilton (1967): (9), (1968): (10), Kekez et al., (1996): (14)]. This property has been used either to lyse cells to recover a metabolite of interest, not naturally excreted by the cell, or to eradicate cells from the environment (disinfecting) or with the aim of non thermal sterilisation of alimentary fluids [Jayaram et al., (1992): (16), Knorr et al., (1994): (17); Qin et al., (1996): (18); Qin et al., (1998): (19)].

This technique has been applied to numerous cell types: bacteria [(Sale and Hamilton, (1967): (8); (Hülsherger et al., (1981, 1983): (11, 12): (Mizuno and Hori, (1988): (13); Jayaram et al., (1992): (16); Grahl and Märkl, (1996): (15); Pothakamory et al., (1996): (26); yeasts [Sale and Hamilton, (1967): (9); Hülsheger et al., (1983): (12); Mizuno and Hori, (1988): (13); Grahl and Märkl, (1996): (15); Gaskova et al., (1996): (28); (Qin et al., (1996): (18), Martin-Belloso et al., (1997); (27)], animal cells [Hamilton and Sale, (1967): (8) Sale and Hamilton, (1968): (10)], and plant cells [Hamilton and Sale, (1967): (8); Sale and Hamilton, (1968): (10); Knorr et al., (1994): (17)]. However, protozoa and in particular free living amoebas have not until now formed the subject matter of such treatments.

Two pulse systems exist, depending on the volume treated: (a) a fixed bed pulse system, known as a batch system, which can only treat small volumes that depend on the dimensions of the electrodes, and (b) a flow pulse system that can treat a flowing cell suspension

The majority of published authors in this field have used the batch system [(9); (10); (11); (12); (13); (16); (28); (26)].

Regarding the flow method, two strategies have been described: continuous flow and sequential flow.

In the sequential flow model, the pulse chamber is filled, the flow is stopped, the field is applied and the chamber is then emptied. This sequential flow model was developed for electromelting work where the contact is mediated by dielectrophoresis.

The advantage of a flow system is the ability to treat large volumes.

The majority of authors have used systems with a field perpendicular to the flow of the solution [(20); (21); (22); (23); (24); (25); (18)] or systems with coaxial electrodes producing a non uniforrn field that is also perpendicular to the flow [(18); (27); (19)].

The Applicants have now developed a method for treating media contaminated with protozoa, by applying a pulsed electric field.

This method can be applied either to a flow or to a static pulse situation. Compared with chlorination and chiotamination, this method for treating colonised media has the particular feature of being a less invasive method as regards the environment.

This method overcomes the disadvantages of known methods, in particular when large volumes are to be treated,

In a first aspect, the invention concepts a method for destroying protozoa, characterized in that a colonised aqueous flow is subjected to an electric field with an intensity of more than 1 kV/cm. Preferably, the intensity varies from 1 to 30 kV/cm, more preferably 1.5 to 15 kV/cm.

The pulse profile can be of the square, exponential decay, trapezoidal, sinusoidal or bipolar wave type.

In these methods, total decontamination of free living amoebas may be obtained in certain aqueous media. Depending on the electrical conditions used, total decontamination of the free living amoebas can be obtained, namely a reduction of the order of 95% in the number of free living amoebas.

The invention also concerns the application of the method for treating a colonised aqueous medium to eliminating protozoa.

The invention will be better understood from the following detailed description made with reference to the accompanying drawings, in which:

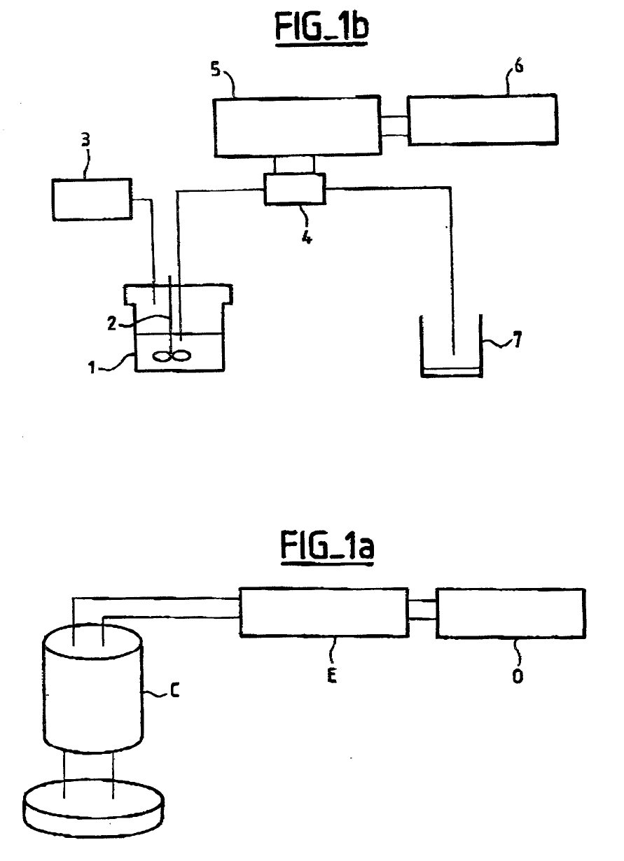

FIG. 1a and 1b are diagrammatic representations of facilities that can be used to carry out the described methods.

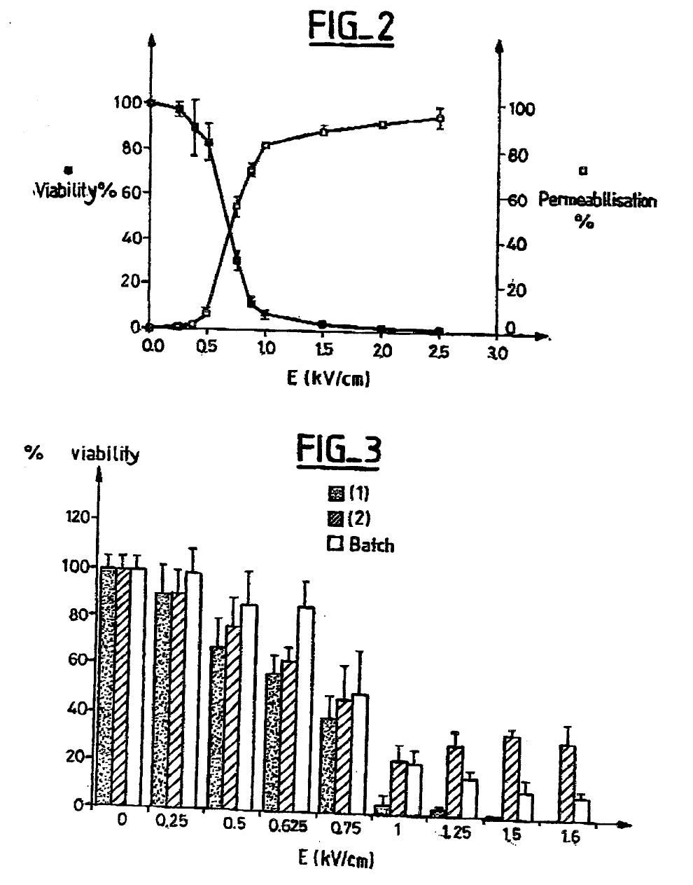

FIG. 2 shows the parallel evolution of the degree of cellular permeabilisation and amoeba viability as a function of the applied electric field intensity.

FIG. 3 shows the percentage of residual viability (illustrating mortality) for different values of field intensity, for orientations parallel and perpendicular to the direction of flow, and for batch systems.

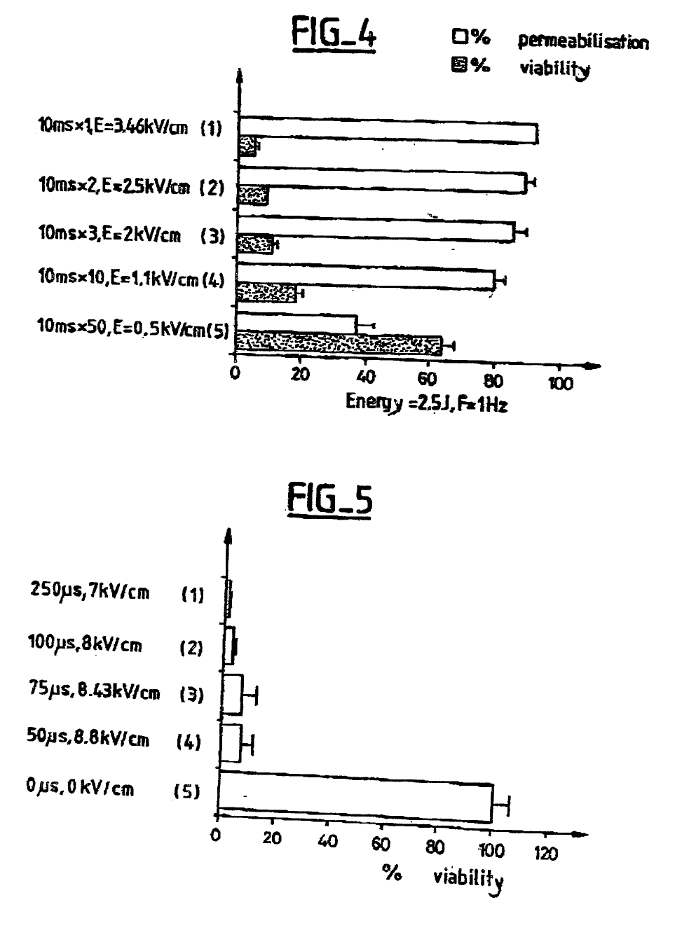

FIG. 4 shows the evolution at a constant energy of 25 J/cm³ of the long term viability and degree of cell permeabilisation.

FIG. 5 illustrates energy optimisation.

The term “colonised medium” as used in the invention means any domestic, natural or industrial aqueous medium that may contain or contains protozoa, in particular amoebas, and particularly free living amoebas, such as natural or heated bathing water, swimming pools and baths, industrial discharges, aqueous media in cooling or heating circuits, aqueous media in ventilation and air conditioning circuits, drinking water, and in general any medium where protozoa, in particular amoebas, and particularly free living amoebas can live, survive or multiply.

The methods of the invention are carried out in continuous flow or sequential flow apparatus.

The number of pulses can vary from 1 to 100 successive pulses, in particular 1 to 50, preferably 1 to 10. The duration can be about 0.5 μs to 24 ms, in particular 1 μs to 10 ms.

The pulse profile can be of the square, an exponential decay, trapezoidal, sinusoidal or bipolar wave type.

The pulses can be delivered at a frequency of 1 to 2000 Hz.

The effective application conditions are determined for intensities of the order of 1.5 kV/cm to 30 kV/cm, for pulses of 0.5 μs to 24 ms and a number of pulses applied to the cell of 1 to 100.

Preferably, ten pulses of 10 ms at 1.5 kV/cm are applied, or one pulse of 10 μs at 11 kV/cm, or one pulse of 50 μs at 9 kV/cm.

### EXAMPLE 1

Experiments were carried out on Naegleria lovaniensis Ar9M1 amoebas. The strain selected was Ar-9M1. It was isolated from warm water (45° C.) from a power station in Florida in 1976 (Stevens et al., 1979). From a phylogenic view point, this species is closest to the pathogenic species N. fowleri; Pernin et al., 1985, showed that N. lovanienesis and N. fowleri are descended from a common ancestor. These two species are both thermophilic and develop particularly well at a temperature of about 44° C. Further, the fact that they have very close isoenzymatic profiles show that they also have similar physiological characteristics. The size of trophozoites of N. locanensis Ar-9M1 was 18.2 μm (8.5-31.5)×10.9 μm (4-21). The cyst diameter was 10.3 μm (7.5-12.5). The size characteristics were close to those of N. fowleri. To work on the vegetative form, the axenic culture was used. In this type of culture, the nutrient elements are not supplied by bacteria but by a nutrient medium. The culture was produced under axenic conditions in plastic trays at 37° C. using Chang culture medium. The pulsing medium was filtered Garonne water with a conductance of the order of 200 μs/cm.

The Generators

The generators used in this study (CNRS and Cober 605P) generated square wave kinetic pulses with a negative polarity. The pulse width could be varied from 5 μs to 24 ms and the application frequency was 0.1 to 10 hz when driven internally and 200 Hz when driven externally. The voltage delivered by the device was 1500 volts maximum )8 A). The Cober 605P was used to deliver intense fields (7 kV/cm≦E<10 kV/cm) by applying a single pulse with a duration of <300 μs. This latter could deliver an outlet voltage of 2 kV (10 A).

Experimental Apparatus

It was placed inside a laminar flow fume cupboard to provide the necessary safety conditions. Viability was evaluated 24 hours after electrical treatment using the crystal violet dye technique.

Cell permeabilisation was quantified using flow cytometry using a naturally non permeating fluorescent marker, propidium iodide.

Batch: The pulse chamber C was constituted by two flat steel plates kept parallel by insulating blocks connected to an electropulser (E) and an oscilloscope (O). The distance 2 between the electrodes was 0.4 cm or 0.25 cm. In FIG. 1a, the arrow indicates cell deposition.

Flow: the pulse system developed in the laboratory was constituted by different elements: a reservoir 1 of cells provided with a stirring means 2, a peristaltic pump 3, a pulse chamber 4 connected to an electropulser 5 and an oscilloscope 6 and a collector system 7 allowing cells to be recovered (see FIG. 1b).

The peristaltic pump (pump, Minipuls 3, Gilson) provided the cell reservoir with an overpressure, to drive the cell suspension towards the electropulsing chamber, without passing between the rollers of the pump. This was provided with a flow measuring system to allow the flow to be accurately adjusted. The flow Q used was based on the concept of residence time such that each cell entering the pulse chamber was subjected to the same electrical conditions. It was defined by the frequency (F), number (N) of pulses and the volume (V) of the pulse chamber by the following relationship:

$$Q ( ml \text{/} minute ) = \frac{frequency ( Hz ) \times 60 \times volume of chamber ( ml )}{Number of pulses applied}$$

Description of Flow Electrodes

1- Field perpendicular to flow: the electrodes were constituted by two parallel steel plates separated by an inter-electrode distance of 0.4 cm. The volume of the pulse chamber was 0.2 ml.

2- Field parallel to flow: the electrodes used were screens, constituted by a mesh (80 μm×100 μm) through which the cells could pass. The inter-electrode distance was 0.93 cm and the volume of the pulse chamber was 0.117 ml.

The electrodes in the two systems were connected to a high tension generator, either CNRS, or COBER 605P, connected to an oscilloscope (Enertec) allowing the electrical parameters supplied to be viewed. The kinetic profile of the pulses delivered by the generator [symbol] flexibility of the electropulser allowed the tension (U), duration (T), number (N) and frequency (F) of the pulses to be adjusted.

It was possible to optimise the method while minimising the energy cost. It has been shown that the loss in viability is not so much connected to the energy supplied by the system during electropulsing but to the manner in which the energy is supplied.

As shown in FIG. 2, the parallel evolution of the level of permeabilisation and loss of viability can be determined as a function of the electric field intensity. The amoebas were electropulsed at different field intensities for ten pulses of 10 ms, delivered at a frequency of 1 Hz. The viability was evaluated 24 hours after electropulsing by the crystal violet dye technique. Permeabilisation was quantified by flow cytometry, by entry of a fluorescent non permeating marker, propidium iodide.

Further, as shown in FIG. 3, the amoeba destruction efficiency (% of viability) by the electric field in the configuration used (batch, field parallel to flow, field perpendicular to flow) were compared. In the three cases, the cells were electropulsed with ten pulses of 10 ms, delivered at a frequency of 1 Hz. The two flow pulse chambers had different volumes, which explains why the flow rates used to provide the same electropulsing conditions were different.

The flow rate in the case where the field was perpendicular to the flow (2-grey) was 1.2 ml/min while that when the field was parallel to flow configuration (1 -black) was 0.71 ml/min.

FIG. 4 shows the change in long term viability ( ) and degree of permeabilisation ( ) at a constant energy of 25 J/cm³.

The intensity of the electric field (E) and total cumulative pulse duration (T) was varied, keeping the value of the product E²×T constant. The pulse duration was arbitrarily fixed at 10 ms and the pulses were delivered at a frequency of 1 Hz. The viability ( ) was revealed at 24 h by the crystal violet dye technique. The permeabilisation ( ) was quantified by flow cytometry, by entry of a non permeating fluorescent marker, propidium iodide. Five electropulsing conditions were employed:

(1) 10 ms×1, E=3.46 kV/cm

(2) 10 ms×2, E=2.5 kV/cm

(3) 10 ms×3, E=2 kV/cm

(4) 10 ms×10, E=1.1 kV/cm

(5) 10 ms×50, E=0.5 kV/cm

The amoebas were more sensitive to the use of short pulses with a high intensity than the use of very long duration, low intensity pulses Amoebas destruction essentially depends on the intensity of the electric field applied and not on the effective pulse duration.

The table below shows, for various field intensities, the pulse time necessary to obtain 95% mortality and the associated energy. It demonstrates that the higher the field intensity, the less energy has to be supplied to the system.

| | | | Power required to |
|---|---|---|---|
| E (kV/cm) | T (μs) | Energy (J/cm³) | treat 1 m³/s (MW) |
| 10.8 | 10 | 0.232 | 0.232 |
| 8.9 | 50 | 0.8 | 0.8 |
| 8 | 100 | 1.3 | 1.3 |
| 7 | 250 | 2.45 | 2.45 |

FIG. 5 shows the results of experiments carried out for fields of up to 8.8 kV/cm under the following conditions:

(1) 250 μs, 7 kV/cm,

(2) 100 μs, 8 kV/cm,

(3) 75 μs, 8.43 kV/cm,

(4) 50 μs, 8.8 kV/cm,

(5) 0 μs, 0 kV/cm

The amoebas were electropulsed at varying electric field intensities for a single short pulse. The viability (% viability) was evaluated 24 hours after electropulsing using the crystal violet dye technique. These parameters obeyed the following law: E=5.4−2.7logT, with T in ms and E in kV/cm, and with T being less than 100 ms.

### REFERENCES

1. Bornhardt J. et al, Biophysik 10:89-98 (1973)

2. Kinosita K. et al., Biochim. Biophys. Acta 554:479-497 (1979)

3. Teissié J. et al., Biochemistry 20: 1548-1554(1981)

4. Ho S. Y. et al., Critical Reviews in Biotechnology, 16: 349-362 (1996)

5. Mir L. M. et al., Experimental Cell Research 175: 15-25(1988)

6. Tsong T. Y., Biophys. J. 60: 297-306 (1991)

7. Hapala I, Critical Reviews In Biotechnology 17: 105-122 (1997)

8. Hamilton W. A. et al., Biochim. Biophys. Acta. 148: 789-800 (1967)

9. Sale J. H. et al., Biochim. Biophys. Acta. 148: 781-788 (1967)

10. Sale J. H. et al., Biochim. Biophys. Acta. 163: 37-43(1968)

11. Hulsheger H. et al., Radiat. Environ. Biophys. 20: 53-65 (1981)

12. Hulsheger H. et al., Radiat. Environ. Biophys. 22: 149-162(1983)

13. Mizuno A. et al., IEEE Transactions on Industry Applications 24: 387-394 (1988)

14. Kekez M. M. et al., Biochimn Biophys. Acta 1278: 79-88 (1996)

15. Gralh T. et al., Appl. Microbiol. Biotechnol. 45: 148-157 (1996)

[symbol]

17. Knorr D. et al, Trends in Food Science and Technology 51: 71-75 (1994)

18. Qin B-L. et al., Critical Reviews in Food Science and Nutrition. 36: 603-627(1996)

19. Qin B-L. et al., IEEE Transactions on Industry Application. 34: 43-50 (1998)

20. Teissié J. et al., Bioelectrochem. Bioenerg. 19: 49-57(1988)

21. Teissié J. et al., Bioelectrochem. Bioenerg. 19: 59-66 (1988)

22. Sixou S. et al., Biochim. Biophys. Acta. 1028: 154-160 (1990)

23. Teissié J. et al., “Charge and Field effects in Biosystems III”, Allen Ed, Birkhauser press pp 449-466 (1992)

24. Rols M. P. et al., Eur. J Biochem. 206: 115-121(1992)

25. Bruggemann U. et al., Transfusion 35: 478-486 (1995)

26. Pothakamury U. R. et al. Journal of Protection 59: 1167-1171(1996)

27. Martin-Bellaos o.m. et al. Journal of Food Processing and Preservation 21: 193-208 (1997)

28. Gaskova et al. Bioelectrochem. Bioenerg. 39: 195-202 (1996).

1. A method for destroying protozoa in an aqueous flow colonized by protozoa, comprising applying a pulsed electric field to the aqueous flow at an intensity from 1 kV/cm to 30 kV/cm.

2. The method according to claim 1, wherein the intensity of the electric field applied to the flow is from 1.5 to 15 kV/cm.

3. The method according to claim 1, wherein said pulsed electric field is applied with a number of 1 to 50 pulses.

4. The method according to claim 1, wherein the flow is continuous.

5. The method according to claim 1, wherein the flow is sequential.

6. The method according to claim 1, wherein the pulsed electric filed is applied in a pulse profile which is of a square wave type, an exponential decay wave type, a sinusoidal wave type, a bipolar wave type or a trapezoidal wave type.

7. The method according to claim 1, wherein the pulsed electric field has pulses at a frequency of 1 Hz to 2000 Hz.

8. The method according to claim 1, wherein the pulsed electric field has pulses with a pulse duration on the order of 1 μs to about 10 ms.

9. The method according to claim 1, wherein the field is substantially parallel to the flow.

10. The method according to claim 1, wherein the field is substantially perpendicular to the flow.

11. The method according to claim 1, wherein the protozoa are amoebas.

12. The method according to claim 11, wherein the protozoa are free living amoebas.

13. A method for eliminating protozoa in an aqueous flow colonized by protozoa, said method comprising applying a pulsed electric field to the aqueous flow at an intensity from 1 kV/cm to 30 kV/cm.
